(19) United States
(12) Patent Application Publication
(10) Pub. No.: US 2009/0326341 A1
(43) Pub. Date: Dec. 31, 2009

(54) APPARATUS FOR MOTOR TRAINING AND EXERCISE OF THE HUMAN BODY

(76) Inventor: Roberto Furlan, Padova (IT)

Correspondence Address: MODIANO & ASSOCIATI, Via Meravigli, 16, MILAN, 20123, IT

(21) Appl. No.: 12/312,335

(22) PCT Filed: Nov. 8, 2007
(86) PCT No.: PCT/EP2007/062036

§ 371 Date: May 6, 2009

(30) Foreign Application Priority Data
Nov. 10, 2006 (IT) PD2006A000413

(51) Int. Cl. A61B 5/00; A63B 71/00

(52) U.S. Cl. 600/301; 482/8

(57) ABSTRACT

An apparatus (1) for motor training and exercise of the human body, comprising:
means (11-21) for acquiring biophysiological data and motion parameters related to a voluntary human action,
means (31-40a) for processing, storing and summarizing the acquired data and parameters,
and means (41-45) for returning at least two feedbacks of information related to the acquired, or acquired and processed, data and parameters, adapted to facilitate correction of the voluntary human action being performed, the information return means being designed to utilize at least one sensory channel.

The present invention relates to an apparatus for motor training and exercise of the human body, particularly for treating disorders of the musculoskeletal system, of the neuromotor system, and disorders linked to cognitive functions (psychological/psychiatric disorders).

### BACKGROUND OF THE INVENTION

The fundamental principles of motor sciences seek to recover corporeity and consider movement as a language of body expression which is destined to affect the psychological and physical growth of a person.

The concept of “psychophysical unity” of the individual is indispensable for the educational process, and reeducational process in the case of the path for recovery from trauma, of a person, especially if one considers the importance of the use and control of one's body in establishing social behaviors and intellectual structures.

To achieve these goals, it is necessary to seek specific learning targets as regards:
physiological enhancement
restoration, consolidation and coordination of basic motor patterns
preparation for sports activity.

Physiological enhancement is to be understood as an improvement of the great vital functions (circulatory and respiratory), of muscle tone and of its subsequent strengthening, and of articular mobility to be maintained and developed over time.

Restoration, consolidation and coordination of basic motor patterns are to be found in the improvement of general postural patterns and of motor patterns, of space-time assessment patterns referred both to oneself and to objects (tools), and of patterns for mental prefiguration of rhythms.

The “basic motor patterns” constitute a pivotal element, which references all the simplest motor skills learned during life: walking, running, balancing, et cetera; the person reaches new, increasingly complex abilities and develops increasingly advanced skills starting from these experiences.

Regular and methodical practice of motor activity, dosed and purposed correctly, is known as exercise when it is intended to increase performance capacity and is known as training when it is intended to learn or relearn, on the part of the individual, postural patterns and motor patterns which are new or not new but at least partly forgotten due to traumas or other disorders.

Exercise and training, in order to be effective, must comply with certain principles:
continuity: constant exercise, avoiding prolonged interruptions,
progressiveness of the load: the quantity and intensity of the exercise cannot remain constant over time, on penalty of risking a loss of effectiveness due to the body that adapts to the load of the exercise,
alternation of work steps and recovery steps: it is important to alternate moments of intense work with others which require more bland commitment,
multilaterality: exercises must be chosen so as to develop harmonically all the factors of performance capacity,
individualization: as a differentiation of the times and methods related to practice, which cannot be equal for all trainees but need to be customized.

Learning correct practice and acquiring specific capabilities is the main goal which one must seek through exercise and training.

There are various forms of learning:
by imitation: when the person, by seeing the exercises performed by others, makes a series of attempts to imitate them,
by intuition: when faced with the need to overcome an obstacle, the person, while not having a model, considers the possible solutions,
by comprehension: it is generally more convenient to learn under the guidance of a good teacher in an intelligent manner.

To allow true learning, the following are important:
motivations: being truly interested in learning is the first condition for success,
understanding the task: to know what one wishes to learn; this is why it is important to observe carefully the demonstration and learn the exact succession of the actions,
repeated executions of the exercise: repetition of the exercise or of a movement is the best way to automate it and therefore acquire skill,
returned information and corrections, i.e., the importance of having feedback to understand how movements just performed were made,
the psychomotor level, i.e., the degree of dexterity possessed by the person who has to learn, a condition which determines the difference between those who learn sooner and those who learn more slowly,
gratification: to be understood as the pleasure derived from the activity and the satisfaction for the results achieved.

As for all other types of teaching, in motor sciences, too, one speaks of customized planning, working in terms of individualization, respecting the different times and different skills that characterize each person.

Individualizing teaching means customizing it according to the problems to be solved, to the skill, capacity, interests and styles of learning of a person, placing the educational purposes in the contest of planned paths.

For correct planning of training and exercise, it is necessary:
to detect the initial situation,
to formulate the specific learning goals,
to choose content in relation to the program,
to choose teaching and methodological strategies,
to select the means and tools to be used,
to identify the methods for verification and assessment of learning.

Devices for motor exercise and training of the human body are currently known.

These devices generally comprise:
a device for acquiring biophysiological data,
means for processing, storing and summarizing the data,
and a device for returning information related to the acquired or acquired and processed data to an expert operator, to whom the training/exercise of the user is entrusted.

Known devices are generally structured to acquire, analyze and return a single feedback or no feedback to the user, whilst data and signals are processed to be subjected to the expert operator, for example a physician or other specialist, who follows the user/patient in the use of the device.

In this way, the user has little or no return information available to understand how the movements that have just been performed were made and to apply the appropriate corrections thereto.

Further, known types of apparatus allow user learning either by intuition, i.e., by possessing the luck/skill to determine autonomously the best solution among the ones that are possible, despite not having a model of the correct postural or motor pattern to be achieved, or by comprehension, i.e., on the basis of the teachings of an operator/teacher; but it is not at all granted that all users have the necessary intuition or that there is an operator/teacher to guide and correct the user constantly, where comprehension of the task is a fundamental step in understanding what has to be learnt and how this learning must occur.

Generally, a user can access one of these known apparatuses only by going to a hospital or hospital-like facility, and if the user is a temporarily or permanently disabled individual, a senior citizen or any other person who has difficulties in moving from home to the place where the apparatus is located, continuing a therapy or a training or exercise process can be very inconvenient if not impossible to do.

### SUMMARY OF THE INVENTION

The aim of the present invention is to provide an apparatus for motor exercise and training of the human body, particularly for treating disorders of the musculoskeletal system, of the neuromotor system, and of disorders which can be linked to the cognitive functions (psychological/psychiatric disorders), which is capable of obviating the limitations of known apparatuses.

Within this aim, an object of the present invention is to provide an apparatus which facilitates and improves learning on the part of the user of how the movement/exercise is to be performed and of how the movement/exercise has actually been performed.

Another object of the present invention is to provide an apparatus which is capable of providing the user, or an expert operator, with the data and parameters that have been collected and processed by means of a plurality of feedbacks, enhancing the sensory channels for perceiving both the proposed movements and the movements actually performed, thus improving learning.

Another object of the present invention is to provide an apparatus which can be managed by an expert operator even remotely.

Another object of the present invention is to provide an apparatus which can be used autonomously by a user even in the absence of an expert operator.

Another object of the present invention is to provide an apparatus which can be manufactured with known systems and technologies.

This aim and these and other objects, which will become better apparent hereinafter, are achieved by an apparatus for motor training and exercise of the human body, characterized in that it comprises:
means for acquiring biophysiological data and motion parameters related to a human action of the type which can be controlled voluntarily by the individual who is performing it,
means for processing, storing and summarizing the acquired data and parameters,
and means for returning at least two feedbacks of information related to the acquired, or acquired and processed, data and parameters, suitable to facilitate correction of the voluntary human action being performed, said information return means being designed to utilize at least one sensory channel.

### BRIEF DESCRIPTION OF THE DRAWINGS

Further characteristics and advantages of the invention will become better apparent from the following detailed description of a preferred but not exclusive embodiment thereof, illustrated by way of non-limiting example in the accompanying drawings, wherein:

FIG. 1 is a schematic view of an apparatus according to the invention,

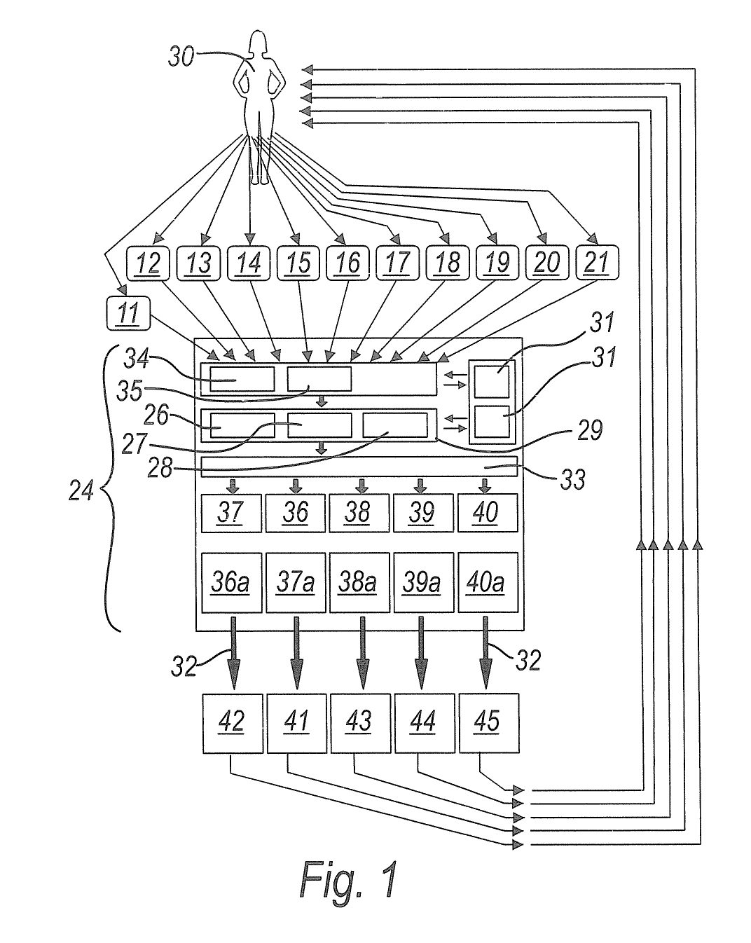

FIG. 2 is a view of an exemplary embodiment of an apparatus according to the invention.

### DESCRIPTION OF THE PREFERRED EMBODIMENTS

With reference to the figures, an apparatus for motor training and exercise of the human body according to the invention is generally designated by the reference numeral 10.

The apparatus 10 comprises
means for acquiring biophysiological data and motion parameters related to a human action of the type which can be controlled voluntarily by the individual who performs said action,
means for processing, storing and summarizing the data and parameters that have been acquired,
and means for returning at least two feedbacks of information related to the acquired, or acquired and processed, data and parameters, suitable to facilitate correction of the voluntary human action being performed, said information return means being designed to utilize at least one sensory channel.

The acquisition means can be constituted by at least one selectively among:
a device 11 for acquiring parameters related to the position and movement of the user or of a part of the body of the user,
a device 12 for acquiring electromyographic data,
a device 13 for acquiring data related to the blood gas saturation level,
a device 14 for acquiring electroencephalographic data,
a device 15 for acquiring data related to respiratory activity and to the exchange of gases in the lungs,
a device 16 for acquiring images,
a device 17 for acquiring data related to cardiac activity and to blood vessel pressure,
a device 18 for acquiring data related to electrodermal activity,
a device 19 for acquiring data related to the noise generated by motion,
a device 20 for acquiring data related to the consumption of glucose,
a device 21 for acquiring temperature data.

The device 11 cited above generically for acquiring parameters related to the position and movement of at least one part of the body of the user can comprise one or more sensors of the electromagnetic and/or ultrasound and/or piezoelectric or variable-resistance type and/or based on systems of the accelerometric type.

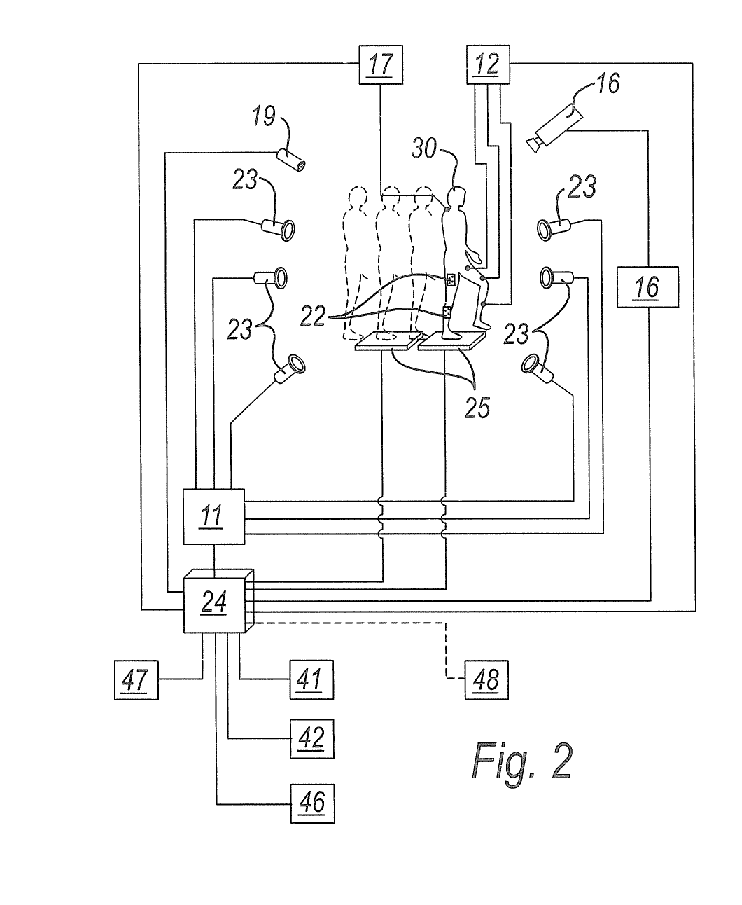

In the exemplary embodiment of FIG. 2, the device 11 for acquiring parameters related to the position and motion of at least one part of the body of the user is of the stereophotogrammetric type with optical markers 22 and image analysis; the position and motion of the markers are detected by a series of infrared video cameras 23, which are connected to the central processing unit 24, described in greater detail hereinafter in its components.

The device 11 for acquiring parameters related to the position and motion of at least one part of the body of the user can also be constituted by a GPS (Global Position System) system over radio frequencies if the user, in order to perform his exercise or training, must travel over relatively large distances or in any case over such distances that other types of sensor would be unable to transmit the detected signals to the receiving base.

In a constructive variation of the invention, the device 11 for acquiring parameters related to the position and motion of at least one part of the body of the user comprises photocells and/or contact switches and/or proximity switches, which may also be managed by electromagnetic emission.

The device 11 for acquiring parameters related to the position and movement of at least one part of the body of the user can also be associated with means for studying body balance, as exemplified in FIG. 2, which illustrates a dynamometric platform 25 on which a user 30 is required to move (walk, run, jump or other similar actions).

The means for studying body balance can be constituted, alternately or additionally, also by one or more stabilometric platforms, force platforms and the like.

The device 11 for acquiring parameters related to the position and motion of at least one part of the body of the user can also be associated with one or more instruments for analyzing the pressure of the sole of the foot or of parts of the body or of accessories connected to parts of the body.

The means for acquiring motion and position parameters can be associated with gymnasium machines, such as for example a stationary bike provided with control over the number of turns of the pedals, or another generic gymnasium machine of the multifunction or single-function type, which is provided with instruments for measuring motion data related to its parts actuated by a user during exercise.

These means for acquiring motion and position parameters can be constituted, as an alternative, by numeric control machines for motor assistance and rehabilitation.

The temperature data acquisition device comprises contact and/or remote probes or, as an alternative, an instrument for reading the invisible spectrum.

The means for processing, storing and summarizing the acquired data and parameters are constituted by the central processing unit 24 and can comprise, associated with a unit 29 for processing the input data and for modulating the outputs,
one or more systems or algorithms 26 for correcting the data with respect to environmental and/or circumstantial interference,
one or more systems 27 for processing signals, data and parameters,
one or more systems 28 for preparing algorithms for analyzing the acquired data.

Such means for processing, storing and summarizing the acquired data and parameters also comprise:
one or more systems 31 for storing signals, acquired data, processed data and parameters, such as a hard disk, a memory chip and the like,
one or more systems (designated schematically by the arrows 32) for data transmission, for example a wired or wireless network,
one or more systems for managing the output of data, which in turn comprise: the algorithms 33 dedicated to the handling of the signals to be sent to appropriately provided hardware cards, said hardware cards, i.e., a video card 36, an audio card 37, and optional cards for tactile feedback 38, for olfactory feedback 39 and for taste feedback 40, and the corresponding software 36a, 37a, 38a, 39a, 40a for managing said cards,
one or more systems 34 for analyzing the signals,
one or more systems 35 for analyzing the data,
one or more user interface systems, of the type for example based on the Windows® system.

Further, the means for processing, storing and summarizing the acquired data and parameters can also comprise at least one selectively among:
one or more systems for providing virtual environments,
one or more systems for virtualizing objects and in general elements which do not exist,
one or more systems for virtual interaction with objects or real shapes or virtual shapes which are stationary or moving, within virtual environments.

The means for returning at least two feedbacks comprise at least two selectively among
means 41 for generating visual feedbacks,
means 42 for generating acoustic feedbacks,
means 43 for generating tactile feedback,
means 44 for generating olfactory feedbacks,
means 45 for generating taste feedback.

Preferably, the delay between the voluntary action and the feedback is such as to allow the user to provide a logical correlation which induces the user to perform a spontaneous correction of the subsequent action or actions.

The faster the feedback, the more the user is facilitated in performing spontaneous correction.

It is also preferable if the indication of the error allows to perceive the correct model or if the correct model is indicated explicitly.

The means 41 for generating visual feedbacks are constituted by at least one among:
a two-dimensional or three-dimensional display screen,
stereoscopic goggles,
a two-dimensional or three-dimensional image projection system,
other similar systems.

The means for generating visual feedbacks can also comprise
a system for displaying multidimensional shapes, including anthropomorphic ones, which are adapted to represent the user, and/or any other interacting virtual or automated individual, and/or any other real interacting individual who is the user of an equivalent system by means of another apparatus,
a system for real-time three-dimensional rendering of objects, optionally also provided with kinematic properties.

The means 42 for generating acoustic feedbacks are constituted by at least one among:
at least one loudspeaker,
at least one monaural system,
mono or stereo headphones,
a positional audio system.

The means 43 for generating tactile feedback are constituted by at least one among:
means of the thermal type,
means of the vibratory type,
means for force or pressure feedback,
a series, a network or an array of electrical stimulators applied to particularly sensitive parts of the body,
air-based or humidified air-based means.

The means of the thermal type are of the type with resistors or Peltier cells or with streams of cold or hot air or the like, while means of the vibratory type can be constituted by sources of vibrations produced by pneumatic or hydraulic pressure waves or eccentric elements actuated by electric motors.

The information return means are adapted to emit feedback to the user 30 of the apparatus and/or to an expert operator to whom the training/exercise of the user is entrusted.

The data processing means are constituted, as mentioned, by a central processing unit 24, which can be controlled directly with an on-site computer 47 or by means of a remote link to a computer or other remote device 46, which is monitored by an expert operator, or for recording the activity of the apparatus 10, a recording which remains available for time-delayed, i.e. not real-time, analysis.

The remote link allows to set up the apparatus 10, for example, even at the user's home, while the expert operator can follow the activities of the user with the apparatus 10 while remaining for example at the clinic or at his/her workplace without having to go to the user to monitor his/her activities.

Remote control can be provided conveniently so that it is performed by means of a data communications network, which is already present in all homes and is available to anyone without excessive additional expenses, or even wirelessly.

The apparatus 10 can also be provided with means for interfacing with one or more portable memories 48, which are available to the user for personalized use of the apparatus 10: the portable memory 48 can contain the training/exercise paths that have been predefined by the expert operator for the user, and the user can perform these activities full autonomously, i.e., without the aid of the presence of such expert operator.

The execution and outcome of the training/exercise session can then be stored in the portable memory 48, available for any use which the user or operator may make of them.

The apparatus 10, in certain configurations, can be enclosed within one or more enclosures which make it ergonomic and convenient to use and carry.

The apparatus 10 according to the invention is well-suited for motor training and exercise of the human body, particularly for treating disorders of the musculoskeletal system, neuromotor system, and disorders which can be linked to the cognitive functions (psychological/psychiatric diseases).

In particular, by way of the possible setup of the apparatus 10 with systems for providing virtual environments or for virtualizing objects and in general elements which do not exist, it becomes possible to approach users affected by disorders of the cognitive functions which can be sensitive to interaction with such virtual environments.

By means of the apparatus 10 it is possible to store the movement sequences to be performed by the user and to propose such recorded movements to the user again so as to induce the user to perform the same movements.

The memories 31 of the processing unit 24 allow, by way of associated systems 28 of analytical algorithms, to select one movement among many, which is assessed by the expert operator or automatically by an appropriately provided algorithm as optimum in order to propose it to the user again.

The apparatus 10 also allows to store, for example by means of a stereophotogrammetric device 11 with optical markers 22, the exact movement of a healthy limb, to be used by reversing it in a mirror-symmetrical manner to propose to the user the optimum movement for the limb with dysfunctions or whose performance is to be improved.

The apparatus 10 also has the advantage of being able to resume and store even very small movements made in the correct direction or in the correct manner and to amplify them with feedback, so that the user has a better perception of the correct direction or of the correct manner in which his/her movement is to proceed.

The apparatus 10 can be programmed and the exercises to be performed can be performed sequentially by the user even in the absence of an expert operator.

The logic of these exercises can be set by the expert operator, while the sequence of exercises is established by one or more of the data analysis systems 28.

As mentioned, to allow true learning, gratification is important, understood as a pleasure deriving from the activity and satisfaction for the results achieved; from this point of view, the apparatus 10 can be provided with a score system, which can be automatically adapted by means of parameters to the performance of the user and is designed to reward the user in proportion to the quality of the exercise performed.

In practice it has been found that the invention thus described solves the problems noted in known types of apparatus for motor training and exercise of the human body, particularly for the treatment of disorders of the musculoskeletal system, neuromotor system, and of disorders which can be linked to the cognitive functions (psychological/psychiatric disorders).

In particular, the present invention provides an apparatus which facilitates and improves learning, on the part of the user, of how the movement/exercise is to be performed and of how the movement/exercise has actually been performed.

Moreover, the present invention provides an apparatus which can provide the user and/or an expert operator with the data and parameters collected and processed by means of a plurality of feedbacks, enhancing the sensory channels for perceiving both the proposed movements and the movements actually performed, thus improving learning.

Further, the present invention provides an apparatus which can be managed by an expert operator even remotely.

Further, the present invention provides an apparatus which can be used autonomously by a user even in the absence of an expert operator.

Moreover, the present invention provides an apparatus which can be manufactured with known systems and technologies.

The invention thus conceived is susceptible of numerous modifications and variations, all of which are within the scope of the appended claims; all the details may further be replaced with other technically equivalent elements.

In practice, the materials employed, so long as they are compatible with the specific use, as well as the dimensions, may be any according to requirements and to the state of the art.

The disclosures in Italian Patent Application No. PD2006A000413 from which this application claims priority are incorporated herein by reference.

What is claimed is:

1-32. (canceled)

33. An apparatus for motor training and exercise of the human body, comprising:
means for acquiring biophysiological data and motion parameters related to a human action of the type which can be controlled voluntarily by the individual who performs it,
means for processing, storing and summarizing the acquired data and parameters,
and means for returning at least two feedbacks of information related to the acquired, or acquired and processed, data and parameters, suitable to facilitate correction of the voluntary human action being performed, said information return means being designed to utilize at least one sensory channel.

34. The apparatus of claim 33, wherein said acquisition means are constituted by at least one, selectively, among:
a device for acquiring parameters related to the position and movement of the user or of a part of the body of the user,
a device for acquiring electromyographic data,
a device for acquiring data related to the blood gas saturation level,
a device for acquiring electroencephalographic data,
a device for acquiring data related to respiratory activity and to the exchange of gases in the lungs,
a device for acquiring images,
a device for acquiring data related to cardiac activity and to blood vessel pressure,
a device for acquiring data related to electrodermal activity,
a device for acquiring data related to the noise generated by motion,
a device for acquiring data related to the consumption of glucose,
a device for acquiring temperature data.

35. The apparatus of claim 34, wherein said device for acquiring parameters related to the position and motion of at least one part of the body of the user selectively comprises: one or more electromagnetic position and motion sensors; or one or more ultrasound position and motion sensors; or a stereophotogrammetric device with optical markers and image analysis; or frequency-based GPS (Global Position System) system; or one or more systems of the accelerometric type; or one or more piezoelectric or variable-resistance sensors; or photocells and/or contact switches and/or proximity switches which can also be managed by electromagnetic emission.

36. The apparatus of claim 34, wherein said device for acquiring parameters related to the position and motion of at least one part of the body of the user is associated with means for studying body balance.

37. The apparatus of claim 36, wherein said means for studying body balance are constituted by a dynamometric platform, by stabilometric platforms, force platforms and the like.

38. The apparatus of claim 34, wherein said device for acquiring parameters related to the position and motion of at least one part of the body of the user is associated with one or more instruments for analyzing the pressure of the sole of the foot or the pressure of portions of the body or of accessories connected to portions of the body.

39. The apparatus of claim 33, wherein said means for acquiring motion and position parameters are associated with gymnasium machines.

40. The apparatus of claim 39, wherein said gymnasium machine, of the multifunction or single-function type, is provided with instruments for measuring the motion data related to its parts actuated by a user.

41. The apparatus of claim 39, wherein said means for acquiring motion and position parameters are associated with numeric-control machines for motion assistance and rehabilitation.

42. The apparatus of claim 34, wherein said device for acquiring temperature data comprises contact and/or remote probes, or an instrument for reading the invisible spectrum.

43. The apparatus of claim 33, wherein said means for processing, storing and summarizing the acquired data and parameters comprise at least one selectively among:
one or more systems for correcting the data with respect to ambient and/or circumstantial noise,
one or more systems for processing the signals, the data and the parameters,
one or more systems for preparing analytical algorithms for the acquired data;
one or more systems for storing signals, acquired data, processed data and parameters,
one or more data transmission systems,
one or more systems for managing the data output,
one or more signal analysis systems,
one or more data analysis systems,
one or more user interface systems;
one or more systems for providing virtual environments,
one or more systems for virtualizing objects and in general elements which do not exist,
one or more systems for virtual interaction with real shapes or objects or virtual shapes which are stationary or moving within virtual environments.

44. The apparatus of claim 33, wherein said means for returning at least two feedbacks comprise at least two selectively among
means for generating visual feedbacks,
means for generating acoustic feedbacks,
means for generating tactile feedback,
means for generating olfactory feedbacks,
means for generating taste feedback.

45. The apparatus of claim 44, wherein said means for generating visual feedbacks are constituted by at least one among:
a two-dimensional or three-dimensional display screen,
stereoscopic goggles,
a two-dimensional or three-dimensional image projection system,
other similar systems.

46. The apparatus of claim 45, wherein said means for generating visual feedbacks can comprise
a system for displaying multidimensional shapes, including anthropomorphic ones, which are adapted to represent said user, and/or any other interacting virtual or automated individual, and/or any other real interacting individual who is the user of an equivalent system by means of another apparatus,
a system for real-time three-dimensional rendering of objects, optionally also provided with kinematic properties.

47. The apparatus of claim 44, wherein said means for generating acoustic feedbacks are constituted by at least one among:
at least one loudspeaker,
at least one monaural system,
mono or stereo headphones,
a positional audio system.

48. The apparatus of claim 44, wherein said means for generating tactile feedback are constituted by at least one among:
means of the thermal type,
means of the vibratory type,
means for force or pressure feedback,
a series, a network or an array of electrical stimulators applied to particularly sensitive parts of the body,
air-based or humidified air-based means.

49. The apparatus of claim 44, wherein said information return means are adapted to emit feedback to the user of the apparatus and/or to an expert operator to whom user training/exercise is entrusted.

50. The apparatus of claim 44, wherein said data processing means are constituted by a central processing unit which can be controlled directly or by remote link.

51. The apparatus of claim 50, wherein said remote control occurs by means of a data communications network which can also be wireless.

52. The apparatus of claim 49, provided with means for interfacing with one or more portable memories which are available to the user for personalized use of the apparatus.
